(19) United States
(12) Patent Application Publication
(10) Pub. No.: US 2010/0240135 A1
(43) Pub. Date: Sep. 23, 2010

(54) System and method for sour gas well testing

(76) Inventors: David M. Seeger, Austin, TX (US); Bonnie S. Davis, Austin, TX (US); Eric C. Klasson, Austin, TX (US); Bryan J. Petrinec, Cedar Park, TX (US)

Correspondence Address: KLAUBER & JACKSON, 411 HACKENSACK AVENUE, HACKENSACK, NJ, 07601, US

(21) Appl. No.: 12/661,583

(22) Filed: Mar. 19, 2010

Related U.S. Application Data
(60) Provisional application No. 61/210,625, filed on Mar. 19, 2009.

(51) Int. Cl. G01N 33/24

(52) U.S. Cl. 436/25; 422/83

(57) ABSTRACT

The invention provides a method of testing sour natural gas by a) removing H₂S from the gas stream, b) converting H₂S to elemental sulfur, and c) recovering elemental sulfur. The invention also provides a substantially mobile system for testing sour natural gas including vehicles allowing the equipment to be operated on surfaces such as the flat beds of trailers. The system features equipment that may be mounted within one or more support structures situated on a mobile vehicle, such as, for instance the flat bed of a trailer. The one or more support structures hold each piece of equipment substantially fixed in place on the vehicles. Each surface such as the flat bed of a trailer may carry one, two or more pieces of equipment. The equipment may include, for instance, an air or oxygen compressor, a knockout vessel, a gas heater, an air heater, a cooler, a molten sulfur separator and a thermal oxidizer.

### FIELD OF INVENTION

This invention relates to flow and pressure testing of natural gas from subterranean wells. Further, the invention relates to the removal and recovery of sulfur which may be present when natural gas flows from sour and other gas wells during the testing of those wells.

### BACKGROUND OF INVENTION

In the oil and gas industry, the producer of oil or gas from a reservoir must have significant knowledge about the size and operating characteristics of that reservoir. This information is required to understand the quality of the individual well, to adequately predict the size of the reservoir, to predict reservoir lifetime, forecast future production, and optimize the production operations. To gain this knowledge, the producer or operator of the field will use various techniques, one of which is well testing. Gathering the maximum amount of reservoir information is critical for the operator in order to optimize the recoverable reserves and well deliverability and have adequate information to make further investment decisions for a particular well. Typically the most important pieces of information gathered during gas well testing are (a) reservoir parameters, (b) flow capacity, (c) gas quality, and (d) estimating the drilling or completion damage (called the skin factor) for the well. This information is critical to the operator or producer of the reservoir or field to optimize the production of the entire field and the particular individual well. The producer may decide to stimulate the well based upon the results of the well test. For example if the well skin was damaged during the drilling process and this is discovered during the well test, then the producer may decide to take steps to stimulate the well. The producer may decide to stimulate the well by fracturing or acidizing the well to improve the skin factor and improve gas production from the well.

During the flow and pressure testing of natural gas wells or oil wells that have associated hydrocarbon gas, hydrogen sulfide (H₂S) is often present in significant quantities, e.g., possibly extending from a few hundreds of parts-per-million-vapor (ppmv) to the mole-percent or volume-percent level (%). In a new application or green field, i.e., one where there is no infrastructure, no pipeline to transport the additional natural gas from the gas test well to the end user, then the natural gas from the gas test must be handled at the site. One of the methods to handle the additional gas from the flow test is to flare the gas; when flaring the gas, any H₂S present in the gas is combusted to form sulfur dioxide (SO₂). The amount of SO₂ that is formed during combustion may be too large to be safely emitted during the test. In addition to the required permitting for the gas well test, the determination of safe SO₂ emission is oftentimes made by each locality, proximity to population centers, personnel on the work crew, etc. The determination of safe levels of SO₂ emission for a well test is not a set standard but rather somewhat discretionary. While there may be no established permissible emission limits for SO₂ during gas well testing, a typical limit for a stationary, constant emission source is 1 long-ton-per-day (tpd) of SO₂. In addition, the Occupational Safety and Health Administration (OSHA) set a Personal Exposure Limit of 5 ppmv for SO₂ for employees in the work place. As an example, a well flowing 25 MMscfd of natural gas that contains 531 ppmv H₂S will produce a flare of 1 tpd of SO₂ emission. For that gas to be flared and achieve a post combustion gas mixture lower than 5 ppmv at ground level or “down wind” of the flare, then the flare off gas must be dispersed or diluted by at least a factor of 106 with surrounding air from the environment. This example appears to describe a situation where the SO₂ may be safely emitted, i.e., a 106-fold dilution rate is most likely achievable using an elevated flare. However, it is desirable to perform well tests where the produced gas contains H₂S in excess of 531 ppmv and to flare that gas with minimal or routine safety considerations on the part of the operators regarding the dispersion of combusted gas into the environment. Keller et al., U.S. Pat. No. 6,946,111 describe one method for removing H₂S from a gas stream, Srinivas et al., U.S. Pat. No. 6,099,819 describes a second method for removing H₂S from a gas stream.

When the SO₂ emission would be too large to be safely emitted, e.g., greater than 1 tpd SO₂ emission, then post-combustion SO₂ or pre-combustion H₂S must be controlled. One approach to reducing the SO₂ emission is to limit the time of the test so as to limit the total amount of SO₂ emitted into the atmosphere. By limiting the time to a few hours, however, the amount of data on flow and reservoir size is also limited, and poor estimates are made for well completion work, pipeline size and gas processing requirements. It would be desirable to provide an alternate approach to flaring sour gas during well testing.

It would be desirable to provide methods and systems whereby sour gas wells may be flow tested for longer periods of time, days to weeks, instead of hours, and as a result of that extended testing, increased information of the flow of the well and the volume of the reserve would be gained. For example, if a gas well or oil well with associated gas is tested such that the flowing gas is 15 MMscfd with 5% H₂S content, flaring that gas will result in the emission of about 57 tpd of SO₂. That amount of SO₂ cannot be safely emitted.

### SUMMARY OF INVENTION

In a first aspect, the invention provides a method of testing sour natural gas by a) removing H₂S from the gas stream, b) converting H₂S to elemental sulfur, and c) recovering elemental sulfur. The H₂S may be removed by reacting the H₂S with oxygen in the presence of a catalyst, preferably at an elevated temperature. The H₂S and oxygen gas may then be cooled causing elemental sulfur vapor to condense as a liquid so that it may be removed from the gas stream. Following that cycle the entire process may be repeated in a following second unit to convert additional H₂S to elemental sulfur, as necessary, before flaring the remaining, now somewhat processed, natural gas. The remaining natural gas may be transported through a thermal oxidizer, or through a modified incinerator, to generate heat. That heat may be used to provide the required heat for the gas as it enters the catalyst reactor earlier in the testing process. The waste gas may then be flared or further treated by an available H₂S scavenging unit. As such, the method of testing sour natural gas may feature a) reacting H₂S with oxygen in the presence of a catalyst, b) cooling the gas to condense the formed elemental sulfur vapor into a liquid, and c) removing the elemental sulfur liquid. The entire process may be repeated once, twice, three or more times in subsequent units to convert additional H₂S to elemental sulfur, as necessary, before d) flaring the remaining, now somewhat processed, natural gas. The remaining natural gas may optionally be first transported through a thermal oxidizer or alternately through a modified incinerator to generate heat. That heat may be used to provide the required heat for the gas as it enters the catalyst reactor(s) earlier in the testing process.

The method of testing sour natural gas may feature a) passing sour natural gas from a well through a valve, b) removing liquids from the gas in a knockout vessel, c) heating the gas to approximately 200-1000° F., preferably 260-840° F., d) passing the gas through an air heater, e) transferring the gas to a catalyst reactor vessel, f) adding oxygen or air to the gas within or prior to the reactor, g) cooling the gas, preferably to about 250-400° F. or 250-280° F., h) separating molten sulfur from the gas, i) removing the molten sulfur in a flash vessel, and optionally j) treating the gas with an additional downstream H₂S removal process followed by flaring the flash gas and all the remaining produced gas. The method may also include k) passing the relatively sulfur-free gas through a thermal oxidizer where a portion of the gas is combusted. The method may further include l) passing the relatively sulfur-free gas back to the natural gas heater or cross heat exchanger to take advantage of the heat evolved through the combustion reaction. The hot gas from the thermal oxidizer may heat the inlet gas, and then the gas may be directed to a flare where it may be combusted. Alternatively, in m), an incinerator may be modified and used in place of the thermal oxidizer to provide the required heat.

In some instances, the sour natural gas is passed from a well where the pressure is reduced to reasonably less than the shut in wellhead pressure allowing for gas flow and subsequent testing of the parameters of the completed well and gas reservoir. The gas pressure may be higher than 300, 400, 500 or 600 psig. In other instances, the gas stream may be tested at less than 200, 250 or 300 psig at the well head. The catalyst reactor temperature may be in the range of 250-840° F. In some embodiments, the temperature of the catalyst reactor vessel is controlled to within approximately +/−50° F., for instance by using internal cooling elements. Also, in some embodiments, the catalyst reactor may include staged internal cooling elements and multiple internal oxygen injection points within the catalyst reactor. The catalyst may be made of any base element of iron, aluminum, titanium, silicon, carbon or silicon carbide, or the oxides of those base elements. In addition the catalyst may be a mixture of one or more of those base metals. Further, the mixture may include one or more of bismuth, sodium, calcium, magnesium, lithium, potassium, vanadium, chromium, manganese, iron, cobalt, silver, sulfur, nickel, copper, niobium, molybdenum, technetium, ruthenium, rubidium, hafnium, tantalum, platinum, palladium, tungsten gold, lead, lanthanum, cerium, praseodymium, selenium, neodymium, promethium, samarium europium, gadolinium, terbium, dysprosium, holmium, erbium, thulium, ytterbium, and lutetium. The catalyst metals may also include mixtures of the base metals or metal oxides, metal sulfides or metal sulfates. The catalyst reactor vessel is preferably designed having dimensions to achieve an actual gas spatial velocity in the range of 250 to 3,000 Hr⁻¹, actual space velocity is defined as the actual volumetric gas flow rate (cubic foot/hr) divided by the volume of the catalyst in the reactor (cubic foot).

In a second aspect, the invention provides a substantially mobile system for testing sour natural gas including vehicles allowing the equipment to be operated on surfaces such as the flat beds of trailers. The system features equipment that may be mounted within one or more support structures situated on a mobile vehicle, such as, for instance the flat bed of a trailer. The one or more support structures hold each piece of equipment substantially fixed in place on the vehicles. Each surface such as the flat bed of a trailer may carry one, two or more pieces of equipment. The equipment may include, for instance, an air or oxygen compressor, a knockout vessel, a gas heater, an air heater, a cooler, a molten sulfur separator and a thermal oxidizer. A heat exchanger for the gas and the catalyst reactor may be located on one trailer, an air/O₂ compressor on a second, a gas cooler and a molten sulfur separator/knockout on a third, a thermal oxidizer and a flare on a fourth and a generator for electricity on a fifth for one reactor set. If a second reactor is required, additional trailers may be provided for the additional heater, reactor, cooler and knockout for that second system. Therefore, a second reactor system may feature one, two or three or more additional vehicles such as a flat bed of a trailer with mounted equipment. In addition to the vehicles provided for transporting the equipment to a test site, one, two or more additional vehicles may be provided for supporting material such as, for instance, interconnecting pipes to interconnect the equipment from one vehicle to the equipment on another, as necessary, cables to interconnect the equipment, controllers, instruments, etc., and any required supports for the interconnecting pipes or cables connecting the equipment between the vehicles. An exemplary aerial view layout of the equipment trailers is shown in FIG. 2.

### BRIEF DESCRIPTION OF THE DRAWINGS

FIG. 1 is a simplified diagram of the process.

FIG. 2 provides a layout of a mobile sour gas well test unit as it may be assembled for operation at a well site.

### DETAILED DESCRIPTION OF THE PREFERRED EMBODIMENTS

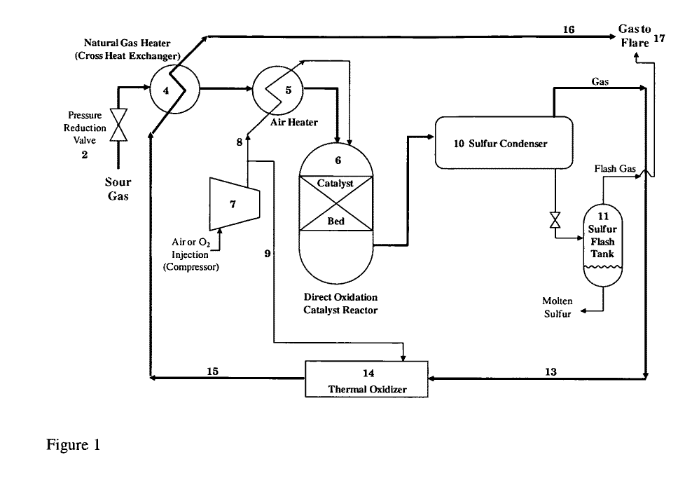

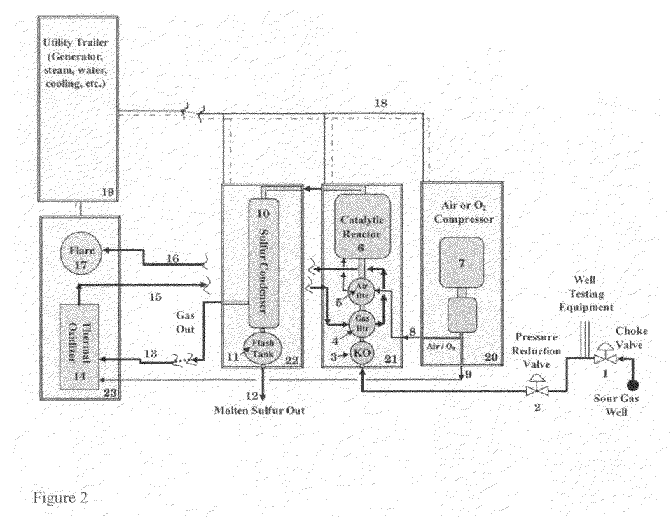

The process and system is described with reference to FIG. 2. The sour natural gas exits the well through choke valve 1 where the gas pressure is controlled for all well testing of parameters of the completed well and the gas reservoir. A second valve, pressure reduction valve 2 as shown in FIGS. 1 and 2, is located downstream of the well testing equipment and is used to control the pressure to the sour gas well testing process equipment. Pressure reduction valve 2 may be located on flat bed trailer 21 as the first element in the sour gas flow path. On trailer 21, after pressure reduction valve 2, the gas passes through piping that leads to knockout vessel 3 where any liquids are removed from the gas stream. After knockout vessel 3, the gas is directed to a natural gas heater or cross heat exchanger 4 where the temperature of the gas is raised. Upon exiting that cross heat exchanger 4, the gas stream flows to an air heater 5 where the required air flow 8 is cross exchanged against the inlet gas flow to take advantage of the heat in the gas and to conserve utility consumption. Once the hot natural gas stream and hot air or oxygen stream exit the air heater 5 they are combined together within the catalyst reactor 6 in one stage or in multiple stages within the catalyst reactor 6. Catalyst reactor 6 may also be located on trailer 21. The air or oxygen is supplied by an air or oxygen compressor 7 located on trailer 20, and the flow is directed to the air or oxygen heater 5 through flow path 8. The air or oxygen required for thermal oxidizer 14 for combustion to generate the heat required for cross heat exchanger 4 is directed through flow path 9. The hot gas exits catalyst reactor 6 and contains sulfur vapor. The hot gas containing sulfur vapor is directed to sulfur condenser 10 located on trailer 22, where the temperature is reduced to condense the sulfur vapor into molten sulfur liquid. The molten sulfur collects by gravity at the bottom of sulfur condenser 10, and the sulfur-free gas exits the top of the sulfur condenser 10. The molten sulfur containing dissolved gas at elevated pressure is then transferred from this separator to flash tank 11 where the pressure is reduced to approximately 5 psig. In flash tank 11 the gas flashes and separates from the molten sulfur, the molten sulfur collects by gravity at the bottom of the flash vessel 11, and the flash gas exits the top of the flash vessel 11 where it is directed via a pipe to flare 17 for disposal via combustion. Molten sulfur 12 is directed out an exit valve at the bottom of the flash vessel 11 to a collection area or heated tankers for use or sale. Sulfur condenser 10 and flash tank 11 are located on trailer 22. After passing through the sulfur condenser 10, the gas passes through pipe 13 to thermal oxidizer 14 where a portion of the gas is combusted and contains a composition made up of primarily carbon dioxide, nitrogen, oxygen, and remaining natural gas. Thermal oxidizer 14 is located on trailer 23. In replacement of the thermal oxidizer 14, an incinerator may be modified and used as the combustion source to provide the necessary heat for the inlet gas cross heat exchanger 4. This gas flows through pipe 15 back to the natural gas heater or cross heat exchanger 4 to take advantage of the heat evolved through the combustion reaction. This stream heats the inlet gas and then flows through pipe 16 to flare 17 to be completely combusted as allowed by local emission standards and general safety concerns. Flare 17 is located on trailer 23. Utility trailer 19 contains the necessary generators that provide electric power for instruments and startup of the equipment. Interconnecting cables and pipes are shown in solid and dashed lines 18, the interconnecting utility lines are shown as lines, also 18, and the interconnecting gas pipes are shown as bold lines between the trailers.

### Process

During a gas well test at a newly drilled well, all the test equipment, pressure monitoring, flow monitoring, etc., is located downstream of a choke valve near the well head. Pressure measurements may also be taken upstream of the choke valve at the wellhead or via downhole pressure instrumentation. The sulfur recovery equipment is located downstream of the choke valve and all well testing equipment. Since the measurement of pressure and flow is critical to well testing, the sulfur control equipment preferably has no substantial back pressure or reduced gas flow effect on the gas during the well test. Therefore, the pressure on the upstream side of the choke valve must be higher than the pressure on the downstream side of the choke valve. The choke valve outlet pipe is preferably larger than the choke valve inlet pipe to avoid choked flow conditions. Choked flow conditions occur when the velocity of gas flowing through a restriction, i.e. a valve, into lower pressure conditions attains sonic (choked) velocity. Such high (sonic) velocity may cause high pipe vibrations, noise, and stress forces. Downstream piping, is preferably sized larger to lower the gas velocity (typically at one-third sonic). In addition, as part of the sulfur control equipment, a knockout vessel may be the first piece of equipment in the gas path of the sour gas well test equipment.

Sour natural gas may exit the well through a choke valve where the pressure is reduced to something reasonably less than the shut in wellhead pressure allowing for gas flow and subsequent testing of the parameters of the completed well and gas reservoir. For example, the gas pressure may be higher than 500 psig. However, in other instances, the gas stream may be tested at less than 300 psig at the well head. In such instances where the gas is less than 300 psig, it must be let down to pressure conditions that are sufficiently below the well head pressure so that the sour gas treatment equipment does not interfere with well testing by causing back pressure.

The choke valve controls the flow of gas during all aspects of well testing, and the well test equipment is located immediately downstream of the choke valve. A second valve, the pressure reduction valve as shown in FIGS. 1 and 2 located downstream of the well testing equipment, may be used to control pressure to the sour gas well testing process equipment. For gas wells operating in excess of about 500 psig, the pressure reduction valve may lower the gas pressure to about 300 psig or less. After the gas pressure is reduced to about 300 psig at the pressure reduction valve, the gas may then pass through piping, e.g. stainless steel, that leads to a knockout vessel where any liquids may be removed from the gas stream. After the knockout vessel, the gas is directed to a natural gas heater where the temperature of the gas is raised to approximately 260-800° F. The temperature may be adjusted depending upon the amount of H₂S in the inlet gas. For example, if the gas contains 2,000 ppmv H₂S, the expected flow rate is 25 MMscfd, and the operator expects to remove enough H₂S so that the resulting flare will only emit 1.0 tpd of SO₂, then the catalyst bed may be operated at a temperature of no less than 409° F. At 300 psig and a temperature below 409° F., sulfur may condense on the surface of the catalyst. In temperature is preferably set at a minimum of 424° F. for a safety factor to avoid the condensation of sulfur on the catalyst surface.

Upon exiting the natural gas heater, the gas stream may flow through piping to an air heater where the required air flow is cross exchanged against the inlet gas flow to take advantage of the heat in the gas and to conserve utility consumption. Using the above example and assuming an inlet gas temperature of 150° F., this natural gas heater requires a heat duty of 7.7 MMBtu/hr. The air compressor, such as a carbon steel air compressor, directs the appropriate amount of either a stream of compressed oxygen or compressed air to the required operating pressure (typically 300 psig, in this case) through piping that may be directed into the shell side of the air heater. Both the natural gas heat exchanger and air heat exchanger may be chosen and size adjusted based upon the requirements encountered. The heat duty depends upon the mass throughput and temperature difference across the exchanger. Once the hot natural gas stream and hot air or oxygen stream exit the air heater, they may be combined together and directed through piping into a reactor catalyst bed. The air or oxygen should preferably not be mixed until immediately upstream of the catalyst reactor bed in the flow path, or mixed at multiple points within the catalyst reactor bed for a staged reaction of H₂S and oxygen. The required amount of oxygen (from air or as oxygen directly) to be added to the gas stream for the reaction in the catalyst bed with H₂S to produce sulfur and water may be calculated according to the reaction equation:

H₂S+½O₂→H₂O+1/nSₙ

Since the design of the reactor is based upon gas pressure, gas flow rate, and gas composition, the amount of H₂S conversion into sulfur is controlled by the amount of added oxygen. For example, natural gas containing 2,000 ppmv H₂S at a 25 MMscfd gas flow rate, 1.0 tpd SO₂ emission at the flare, 300 psig gas pressure and 424° F. at the catalyst reactor bed, 1,549 lb/day of O₂ (on a molecular basis) should be added to the process. Assuming the air added to the natural gas stream is 100° F., it should be heated to 424° F. through the air heat exchanger. With a required O₂ rate of 1,549 lb/day, it follows that 6,648 lb/day of air is added to the system (i.e. 21% O₂/79% N₂) and equates to a required heat duty of 22,273 Btu/hr.

The reactor as well as the internal supports for the catalyst bed are preferably constructed of stainless steel since H₂S is corrosive. The catalyst reactor bed also preferably has internal cooling elements that maintain the temperature within the reaction zone. Preferably, the temperature is controlled to within approximately +/−50° F., as the H₂S oxidation reaction and other side hydrocarbon oxidation reactions that may occur in the reactor are exothermic and may cause significant temperature increases within the reactor. The amount of intercooling required may be calculated based upon the heat of reaction of H₂S oxidation and other side hydrocarbon reactions. For example, at 25 MMscfd of natural gas flow and 2,000 ppmv H₂S, the heat duty of the reactor is approximately 7.7 MMBtu/hour and the temperature rise across the catalyst bed is approximately 5° F. Controlling the temperature aids in controlling unwanted side reactions such as hydrocarbon oxidation while optimizing the selective conversion of H₂S into sulfur. Temperature control also preserves the mechanical integrity of the vessel thus avoiding fracturing while operating at elevated temperatures and pressures.

The catalyst may be made of any base element of iron, aluminum, titanium, silicon, carbon or silicon carbide, or the oxides of those base elements. In addition the catalyst may be a mixture of one or more of those base metals. The catalyst may be based on zeolites. Further, the catalyst may include one or more of bismuth, sodium, calcium, magnesium, lithium, potassium, vanadium, chromium, manganese, iron, cobalt, silver, sulfur, nickel, copper, niobium, molybdenum, technetium, ruthenium, rubidium, hafnium, tantalum, platinum, palladium, tungsten, gold, lead, lanthanum, cerium, praseodymium, selenium, neodymium, promethium, samarium, europium, gadolinium, terbium, dysprosium, holmium, erbium, thulium, ytterbium, and lutetium. The catalyst metals may also include mixtures of the base metals or metal oxides, metal sulfides or metal sulfates.

The catalyst reactor vessel is preferably designed having dimensions to achieve a spatial velocity in the range of 250 to 3000 Hr⁻¹, based on actual conditions. Spatial velocity is calculated based upon gas flow rates at operating temperature and pressure. For example, if the gas rate is 25 MMscfd at an inlet temperature of 424° F. and pressure of 300 psig, then the volume of catalyst used in the reactor bed is approximately 5.5 feet in diameter and 6.0 feet in height, excluding the internal cooling elements, if any. At these dimensions, the actual gas spatial velocity through the catalyst material is 581 Hr⁻¹, not including the added air.

The hot gas exits the catalyst reactor containing sulfur vapor. The hot gas containing sulfur vapor may be directed to a gas cooler such as a stainless steel gas cooler where the temperature may be reduced to about 250-280° F. as appropriate in order to condense the sulfur vapor into molten sulfur liquid. The cooler may be chosen based upon the required heat duty of the exchanger depending upon the gas flow rate and temperature difference across the cooler. For example, assuming the temperature of the 25 MMscfd of gas exiting the reactor is at 429° F. and is cooled to 265° F., the required heat duty is approximately 4.7 MMBtu/hr. The velocity of the gas through the cooler is also an important factor in the design and operation. Successful operation of sulfur condensers are dependent on the density (ρ) and velocity (v) of the gas so that ρv2=600 or less. At values above 600, a supersaturation condition (fogging) may occur causing the condenser to over perform.

The gas-molten sulfur mixture then passes into a molten sulfur separator, preferably of stainless steel, where the molten sulfur collects by gravity at the bottom of the vessel and the sulfur-free gas exits the top of the vessel. Depending upon conditions and the remaining H₂S concentration in the gas leaving this vessel, another stainless steel gas heater, air/O₂ injection gas heater, catalyst reactor, gas cooler, molten sulfur separator and flash tank system may be required to further reduce the H₂S concentration in the gas. The molten sulfur with dissolved gas at approximately 300 psig is then transferred from the molten sulfur separator through a flash valve where the pressure is reduced to approximately 5 psig. If the pressure drop across this valve results in significant cooling of the molten sulfur (temperature below 250° F.), then an additional heater or a steam jacketed valve may be required. The molten sulfur with dissolved gas passes through the valve where the gas flashes and separates from the molten sulfur. The molten sulfur collects by gravity at the bottom of the flash tank, and the flash gas exits the top of the flash tank where it is directed via piping to the flare for disposal via combustion. The molten sulfur is directed out an exit valve at the bottom of the flash tank to a collection area or heated tankers.

After passing through the last molten sulfur separator, the gas passes through piping to a thermal oxidizer where a portion of the gas is combusted. The substantially processed gas contains primarily carbon dioxide, nitrogen, oxygen, and un-combusted natural gas at this point. This substantially processed gas may flow through piping back to the natural gas heater or cross heat exchanger to take advantage of the heat evolved from the combustion reaction. This stream heats the inlet gas by cross-exchanging of heat and then flows through piping to a flare to be fully combusted as allowed by local emission standards and general safety concerns.

### System

Mobilizing a sour gas well test unit requires providing vehicles that allow the equipment to be operated on surfaces such as the flat beds of trailers. On each trailer the equipment is mounted within a support structure holding that piece of equipment substantially fixed in place on the trailer. Each surface such as a trailer may carry one, two or more pieces of equipment. For example, a heat exchanger for the gas and the catalyst reactor may be located on one trailer, an air/O₂ compressor on a second, a gas cooler and a molten sulfur separator and flash tank on a third, a thermal oxidizer and a flare on a fourth and a generator for electricity on a fifth for one reactor set. If a second reactor is required, additional trailers are required for the additional heater, reactor, cooler and knockout for that second system. Therefore, a second reactor system requires approximately two or three additional trailers with mounted equipment. In addition to the trailers that transport the equipment to a test site, one or two additional trailers may be required for supporting material such as: a) interconnecting pipes to interconnect the equipment from one flatbed trailer to the equipment on another, as necessary, b) cables to connect the equipment/controllers/instruments/etc., and c) any required supports for the interconnecting pipes or cables connecting the equipment between the trailers. A possible aerial view layout of the equipment trailers is shown in FIG. 2. FIG. 2 does not depict support trailers that carry the interconnecting cables and pipes. In FIG. 2, the interconnecting cables are shown as dashed lines while the interconnecting utility lines are shown as solid lines. Interconnecting gas pipes are shown as bold solid lines between the trailers.

The routine equipment such as knockout vessels, separators, flash vessels, flares, interconnecting pipes, assembly of all equipment into skids, wiring, vessel mounting onto skids, etc., may be obtained from but not limited to such fabrication companies as: Flint Energy Services Ltd. of Calgary, Alberta; Toromont, Concord, Ontario; Ortloff, Midland, Tex.; Gulf Coast Welding Corp., Houston, Tex. Air or oxygen compressors are available from but not limited to: Universal Compressor, Gardena, Calif.; Ariel Corp, Mount Vernon, Ohio; or Praxair, Danbury, Conn. Heat exchangers are available from but not limited to: Ametek, Paoli, Pa. or Alfa Laval, Lund, Sweden. Thermal oxidizers may be obtained from but not limited to: HiTemp Technology Corporation, Flemington, N.J. A suitable reactor is available from but not limited to: Lurgi AG, Frankfurt am Main, Germany or Linde AG, Munich, Germany. An incinerator may be available from but not limited to Questor Technology, Inc., Calgary, Alberta.

### EXAMPLES

### Example 1

### High Pressure Well Test

If the gas well pressure is above 300 psig at the well head, upstream of the choke valve, then the pressure will be reduced across the choke valve so that the Mobile Sour Gas Well Test Unit will operate at 300 psig downstream of the choke valve. The gas contains 2,000 ppmv H₂S. The gas flow rate is 25 MMscfd, the operating pressure of the sour gas control equipment is 300 psig, and the operator expects to remove enough H₂S so that the resulting flare will only emit 1.0 tpd of SO₂. The catalyst bed is operated at 424° F. to prevent sulfur condensation on the surface of the catalyst. The catalyst bed operating temperature is dependent upon the inlet gas stream composition and will be adjusted as necessary. If the inlet gas temperature is 150° F., the natural gas heater requires a heat duty of 7.7 MMBtu/hr. To convert the required amount of H₂S into elemental sulfur, 1,549 lb/day of O₂ (molecular basis) is added to the gas upstream of the catalyst reactor bed or 6,648 lb/day of air (i.e. 21% O₂/79% N₂), the required heat duty for that air stream is 22,273 Btu/hr. The reactor does not have internal cooling. The temperature rise across the catalyst bed is approximately 5° F. The reactor bed is filled with approximately 6,272 lb of catalyst and has approximate dimensions of 5.5 feet diameter and 6.0 feet height. The gas temperature exiting the reactor is at 429° F. and is cooled to 265° F. The required heat duty is approximately 4.7 MMBtu/hr. The molten sulfur is separated from the gas stream in the separator. The natural gas is directed to a thermal oxidizer where the necessary amount of gas is combusted to generate the required heat for the utilities of the process described above.

### Example 2

### Low Pressure Well Test

If the gas well pressure is below 300 psig at the well head, upstream of the choke valve, then the pressure is reduced across the choke valve so that the Mobile Sour Gas Well Test Unit will operate at near atmospheric pressure downstream of the choke valve. Gas at the wellhead is approximately 100 psig, and the operation of the catalyst reactor is at 50 psig. The gas contains 2,000 ppmv H₂S, the gas flow rate is 25 MMscfd, the operating pressure of the sour gas control equipment is 50 psig, and the operator expects to remove enough H₂S so that the resulting flare will only emit 1.0 tpd of SO₂. The catalyst bed is operated at 370° F. to prevent sulfur condensation on the surface of the catalyst. If the inlet gas temperature is 150° F., the natural gas heater requires a heat duty of 6.0 MMBtu/hr. To convert the required amount of H₂S into elemental sulfur, 1,549 lb/day of O₂ (molecular basis) is added to the gas upstream of the catalyst reactor bed or 6,648 lb/day of air (i.e. 21% O₂/79% N₂), the required heat duty for that air stream is 18,548 Btu/hr. The reactor does not have internal cooling. The temperature rise across the catalyst bed is approximately 5° F. The reactor bed is filled with approximately 30,800 lb of catalyst and has approximate dimensions of 9.0 feet diameter and 11.0 feet height. The gas temperature exiting the reactor is at 375° F. and is cooled to 265° F. The required heat duty is approximately 3.1 MMBtu/hr. The molten sulfur is separated from the gas stream in the separator. The natural gas is directed to a thermal oxidizer where the necessary amount of the gas is combusted to generate the required heat for the utilities of the process as described above.

### Example 3

### High Pressure, High H₂S Content Well Test

If the gas well pressure is above 300 psig at the well head, upstream of the choke valve, then the pressure is reduced across the choke valve so that the Mobile Sour Gas Well Test Unit operates at 300 psig downstream of the choke valve. The gas contains 5% H₂S, the gas flow rate is 15 MMscfd, the operating pressure of the sour gas control equipment is 300 psig, and the operator expects to remove enough H₂S so that the resulting flare will emit 5.0 tpd of SO₂. The catalyst bed is operated at 635° F. to prevent sulfur condensation on the surface of the catalyst. If the inlet gas temperature is 150° F., the natural gas heater requires a heat duty of 9.3 MMBtu/hr. To convert the required amount of H₂S into elemental sulfur, 28,823 lb/day of O₂ (molecular basis) is added to the gas upstream of the catalyst reactor bed or 123,746 lb/day of air (i.e. 21% O₂/79% N₂). The required heat duty for that air stream is 688,798 Btu/hr. The reactor has internal cooling, and the heat evolved from the reactor is approximately 1.9 MMBtu/hr. The temperature rise across the catalyst bed would be approximately 109° F., however the reactor has internal cooling elements to maintain operating temperature of approximately 635° F. The reactor bed is filled with approximately 5,227 lb of catalyst and has approximate dimensions of 5.5 feet diameter and 5.0 feet height, not including the internal cooling elements. The gas temperature exiting the reactor is approximately 635° F. and is cooled to 265° F. in the sulfur condenser. The required heat duty is approximately 7.8 MMBtu/hr. The molten sulfur is separated from the gas stream in the separator. The natural gas is directed to a thermal oxidizer where the necessary amount of the gas is combusted to generate the required heat for the utilities of the process as described above.

### Example 4

### Low Pressure, High H₂S Content Well Test

If the gas well pressure is below 300 psig at the well head, upstream of the choke valve, then the pressure is reduced across the choke valve so that the Mobile Sour Gas Well Test Unit operates at near atmospheric pressure downstream of the choke valve. The gas at the wellhead is approximately 100 psig, and the operation of the catalyst reactor is at 50 psig. The gas contains 5% H₂S, the gas flow rate is 15 MMscfd, the operating pressure of the sour gas control equipment is 50 psig, and the operator expects to remove enough H₂S so that the resulting flare will only emit 5.0 tpd of SO₂. The catalyst bed must be operated at a temperature of 533° F. at the inlet to the reactor to prevent sulfur condensation on the surface of the catalyst. If the gas temperature at the inlet to the inlet gas heater is 150° F., the natural gas heater requires a heat duty of 7.1 MMBtu/hr. To convert the required amount of H₂S into elemental sulfur, 28,823 lb/day of oxygen is added to the gas upstream of the catalyst reactor bed or 123,746 lb/day of air (i.e. 21% O₂/79% N₂), the required heat duty for that air stream is 549,095 Btu/hr. The reactor does not have internal. The temperature rise across the catalyst bed is approximately 109° F. The reactor bed is filled with approximately 23,800 lb of catalyst and has approximate dimensions of 9.0 feet diameter and 8.5 feet height. The gas temperature exiting the reactor is at 642° F. and is cooled in the sulfur condenser to 265° F. The required heat duty is approximately 7.8 MMBtu/hr. The molten sulfur is separated from the gas stream in the separator. The natural gas is directed to a thermal oxidizer where the necessary amount of the gas is combusted to generate the required heat for the utilities of the process as described above.

We claim:

1. A method of testing sour natural gas comprising a) removing H₂S from the gas stream, b) converting H₂S to elemental sulfur, and c) recovering elemental sulfur.

2. The method of claim 1 wherein H₂S is removed by reacting the H₂S with oxygen in the presence of a catalyst at an elevated temperature.

3. The method according to claim 1 further comprising cooling the formed elemental sulfur vapor so that the elemental sulfur vapor condenses as a liquid.

4. The method according to claim 1 further comprising transporting the remaining natural gas through a thermal oxidizer to generate heat.

5. A method of testing sour natural gas comprising a) reacting H₂S with oxygen in the presence of a catalyst, b) cooling the gas to condense elemental sulfur vapor and form a liquid, and c) removing the elemental sulfur vapor.

6. The method according to claim 5 further comprising d) flaring the remaining natural gas.

7. A method of testing sour natural gas comprising
a) passing sour natural gas from a well through a valve;
b) removing liquids from the gas in a knockout vessel;
c) heating the gas to 260-840° F.;
d) passing the gas through an air heater;
e) transferring the gas to a reactor catalyst vessel;
f) cooling the gas to about 250-280° F.;
g) separating molten sulfur from the gas;
h) removing the molten sulfur in a flash vessel; and
i) flaring flash gas.

8. The method according to claim 7 further comprising j) passing the relatively sulfur-free gas through a thermal oxidizer where the gas is combusted to below emission standards.

9. The method according to claim 7 further comprising k) passing the relatively sulfur-free gas back to the heater or cross heat exchanger to take advantage of the heat evolved through the combustion reaction.

10. The method according to claim 7 wherein the sour natural gas is passed from a well where the pressure is reduced to reasonably less than shut in wellhead pressure.

11. The method according to claim 7 wherein the gas pressure in step a) is greater than 300 psig.

12. The method according to claim 7 wherein the gas pressure in step a) is less than 300 psig.

13. The method according to claim 7 the temperature of the catalyst reactor vessel is controlled to within approximately +/−50° F.

14. The method according to claim 7 wherein the catalyst is selected from the group consisting of iron, aluminum, titanium, silicon, carbon, silicon carbide, or oxides thereof, bismuth, sodium, calcium, magnesium, lithium, potassium, vanadium, chromium, manganese, iron, cobalt, silver, sulfur, nickel, copper, niobium, molybdenum, technetium, ruthenium, rubidium, hafnium, tantalum, platinum, palladium, tungsten gold, lead, lanthanum, cerium, praseodymium, selenium, neodymium, promethium, samarium europium, gadolinium, terbium, dysprosium, holmium, erbium, thulium, ytterbium, and lutetium or is based on the mineral zeolite.

15. The method according to claim 7 wherein the catalyst reactor vessel provides a spatial velocity in the range of 250 to 3000 Hr⁻¹.

16. A substantially mobile system for testing sour natural gas comprising at least one mobile vehicle, at least one support structure situated on a mobile vehicle, an air or oxygen compressor, a knockout vessel, a gas heater or cross heat exchanger, an air heater, a cooler, a molten sulfur separator and a thermal oxidizer.

17. A substantially mobile system for testing sour natural gas according to claim 16 further comprising a second reactor.

18. A substantially mobile system for testing sour natural gas according to claim 16 further comprising interconnecting pipes to interconnect equipment from one vehicle to equipment on another vehicle.

19. A substantially mobile system for testing sour natural gas according to claim 16 further comprising one or more controllers in communication with one another.

20. A substantially mobile system for testing sour natural gas according to claim 16 wherein the mobile vehicle is a flatbed truck.
